(19) United States
(12) Patent Application Publication
(10) Pub. No.: US 2004/0181214 A1
(43) Pub. Date: Sep. 16, 2004

(54) Passively cooled array

(76) Inventors: Robert J. Garabedian, Tyngsboro, MA (US); Amy C. Kelly, San Francisco, CA (US); Steven K. Landreville, Mountain View, CA (US)

Correspondence Address: BINGHAM, MCCUTCHEN LLP, THREE EMBARCADERO, SUITE 1800, SAN FRANCISCO, CA, 94111-4067, US

(21) Appl. No.: 10/387,812

(22) Filed: Mar. 13, 2003

(51) Int. Cl. A61B 18/18

(52) U.S. Cl. 606/41; 607/101

(57) ABSTRACT

A tissue ablation system includes an elongated shaft, such as a surgical probe shaft, and an needle electrode array mounted to the distal end of the shaft, and an ablation source, such as, e.g., a radio frequency (RF) generator, for providing ablation energy to the electrode array. The tissue ablation system further includes a heat sink disposed within the distal end of the shaft in thermal communication with the needle electrode array. In this manner, thermal energy is drawn away from the needle electrode array, thereby cooling the electrode array and providing a more efficient ablation process. The tissue ablation system further comprises a coolant flow conduit in fluid communication with the heat sink, so that the thermal energy can be drawn away from the heat sink. In the preferred embodiment, the flow conduit includes a thermal exchange cavity in fluid communication with the heat sink, a cooling lumen for conveying a cooled medium (such as, e.g., saline at room temperature or below) to the thermal exchange cavity, and a return lumen for conveying a heated medium from the thermal exchange cavity. The tissue ablation system further comprises a pump assembly for conveying the cooled medium through the cooling lumen to the thermal exchange cavity at the distal end of the shaft.

### FIELD OF THE INVENTION

The field of the invention relates generally to the structure and use of radio frequency (RF) electrosurgical probes for the treatment of tissue, and more particularly, to electrosurgical probes having multiple tissue-penetrating electrodes that are deployed in an array to treat large volumes of tissue.

### BACKGROUND OF THE INVENTION

The delivery of radio frequency (RF) energy to target regions solid tissue is known for a variety of purposes of particular interest to the present inventions. In one particular application, RF energy may be delivered to diseased regions (e.g., tumors) in target tissue for the purpose of tissue necrosis. RF ablation of tumors is currently performed within one of two core technologies.

The first technology uses a single needle electrode, which when attached to a RF generator, emits RF energy from the exposed, uninsulated portion of the electrode. This energy translates into ion agitation, which is converted into heat and induces cellular death via coagulation necrosis. In theory, RF ablation can be used to sculpt precisely the volume of necrosis to match the extent of the tumor. By varying the power output and the type of electrical waveform, it is possible to control the extent of heating, and thus, the resulting ablation. The diameter of tissue coagulation from a single electrode, however, has been limited by heat dispersion. As a result, multiple probe insertions have been required to treat all but the smallest lesions. This considerably increases treatment duration and requires significant skill for meticulous precision of probe placement.

Increasing generator output has been unsuccessful for increasing lesion diameter, because an increased wattage is associated with a local increase of temperature to more than 100° C., which induces tissue vaporization and charring. This then increases local tissue impedance, limiting RF deposition, and therefore heat diffusion and associated coagulation necrosis. To reduce the local temperature, thereby minimizing tissue vaporization and charring, the needle electrode is cooled. Specifically, two coaxial lumens are provided in the needle electrode, one of which is used to deliver a cooled saline (e.g., room temperature or cooler) to the tip of the electrode, and the other of which is used to return the saline to a collection unit outside of the body. See, e.g., Goldberg et al., Radiofrequency Tissue Ablation: Increased Lesion Diameter with a Perfusion Electrode, Acad Radiol, August 1996, pp. 636-644.

The second technology utilizes multiple needle electrodes, which have been designed for the treatment and necrosis of tumors in the liver and other solid tissues. PCT application WO 96/29946 and U.S. Pat. No. 6,379,353 disclose such probes. In U.S. Pat. No. 6,379,353, a probe system comprises a cannula having a needle electrode array reciprocatably mounted therein. The individual electrodes within the array have spring memory, so that they assume a radially outward, arcuate configuration as they are advanced distally from the cannula. In general, a multiple electrode array creates a larger lesion than that created by an uncooled needle electrode. Current electrode array manufacturers, however, do not include cooling within their designs, and subsequently have to be concerned about charring, and its interference with the operation of the electrode array.

Thus, there is a need for an improved cooling assembly for a multiple electrode array that provides for a more efficient and effective ablation treatment of tissue.

### SUMMARY OF THE INVENTION

The present inventions use heat sinks and coolant flow conduits to provide cooling to needle electrodes used by medical probe assemblies and systems for efficiently ablating tissue.

In accordance with the present inventions, a medical probe assembly for ablating tissue comprises an elongated shaft, one or more needle electrodes extending from the distal end of the shaft, a heat sink disposed within the distal end of the shaft in thermal communication with the needle electrode(s), and a coolant flow conduit disposed within the shaft in fluid communication with the heat sink. In the preferred embodiment, the elongated shaft is a surgical probe shaft. In its broadest aspects, however, the present inventions should not be limited to surgical probe shaft, but contemplate other types of elongated probe shafts, such as catheter shafts. In the preferred embodiment, an array of needle electrodes extend from the distal end of the shaft. An optional core member, around which the needle electrodes are circumferentially disposed, can also extend from the distal end of the shaft. The one or more needle electrodes can be directly or indirectly connected to an ablation source. For example, if the ablation source is a radio frequency (RF) ablation source, the proximal ends of the needle electrodes can be coupled to the ablation source, or intermediate electrical conductors, such as, e.g., RF wires or the elongate shaft itself, can be used to couple the proximal ends of the needle electrodes to the ablation source.

The heat sink can be configured in any particular manner that thermally draws heat away from the one or more electrodes. For example, the heat sink can be composed of a solid material to provide for a maximum thermal energy absorbing capability. Alternatively, the heat sink can comprise a sealed cavity containing a medium that transitions from a liquid state to a gaseous state when heated, and transitions from the gaseous state back to the liquid state when cooled. As a result, the state transition of the medium absorbs quickly absorbs heat from the heat sink. The internal air pressure within the sealed cavity is preferably less than the air pressure external to the cavity to hasten the transition of the medium from the liquid state to the gaseous state. A wicking material can be disposed within the sealed cavity, so that the transition of the medium from the liquid state to the gaseous state, and from the gaseous state back to the liquid state, can be accomplished in a more controlled and stable manner.

The coolant flow conduit can be configured in any particular manner that thermally draws thermal energy away from the heat sink. For example, in the preferred embodiment, the coolant flow conduit comprises a cooling lumen for conveying a cooled medium from the proximal end of the shaft to the heat sink, and a return lumen for conveying a heated medium from the heat sink to the proximal end of the shaft. The exemplary coolant flow conduit also comprises a thermal exchange cavity in fluid communication between the cooling and return lumens and the heat sink. The cooling and return lumens can be formed by disposing an inner tube with the shaft. In this case, one of the cooling lumen and return lumen is formed within the inner tube, and the other of the cooling lumen and return lumen is an annular lumen that is formed between the inner surface of the shaft and the outer surface of the inner tube. Alternatively, the cooling and return lumens can be disposed in a side-by-side relationship, rather than in a coaxial relationship.

In the preferred embodiment, the medical probe assembly comprises a cannula having a central lumen in which the shaft is reciprocally disposed. In this manner, the needle electrode(s) can be conveniently delivered to and deployed within a tissue to be treated. The medical probe assembly can be used with an ablation source, such as, e.g., a radio frequency (RF) ablation source, to provide ablation energy to the needle electrode(s). The medical probe assembly can also be used with a pump assembly, which conveys the cooled liquid medium through the cooling lumen of the medical probe assembly.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a tissue ablation system constructed in accordance with one preferred embodiment of the present inventions;

FIG. 2 is a partially cutaway cross-sectional view of a probe assembly used in the tissue ablation system of FIG. 1, wherein a needle electrode array is particularly shown deployed from the probe assembly;

FIG. 3 is a partially cutaway cross-sectional view of the probe assembly used in the tissue ablation system of FIG. 1, wherein the needle electrode array is particularly shown retracted within the probe assembly;

FIG. 4 is a partially cut-away cross-sectional view of an alternative embodiment of a heat sink used in the probe assembly of FIGS. 2 and 3; and

FIGS. 5A-5D illustrates cross-sectional views of one preferred method of using the tissue ablation system of FIG. 1 to treat tissue.

### DETAILED DESCRIPTION OF THE PREFERRED EMBODIMENTS

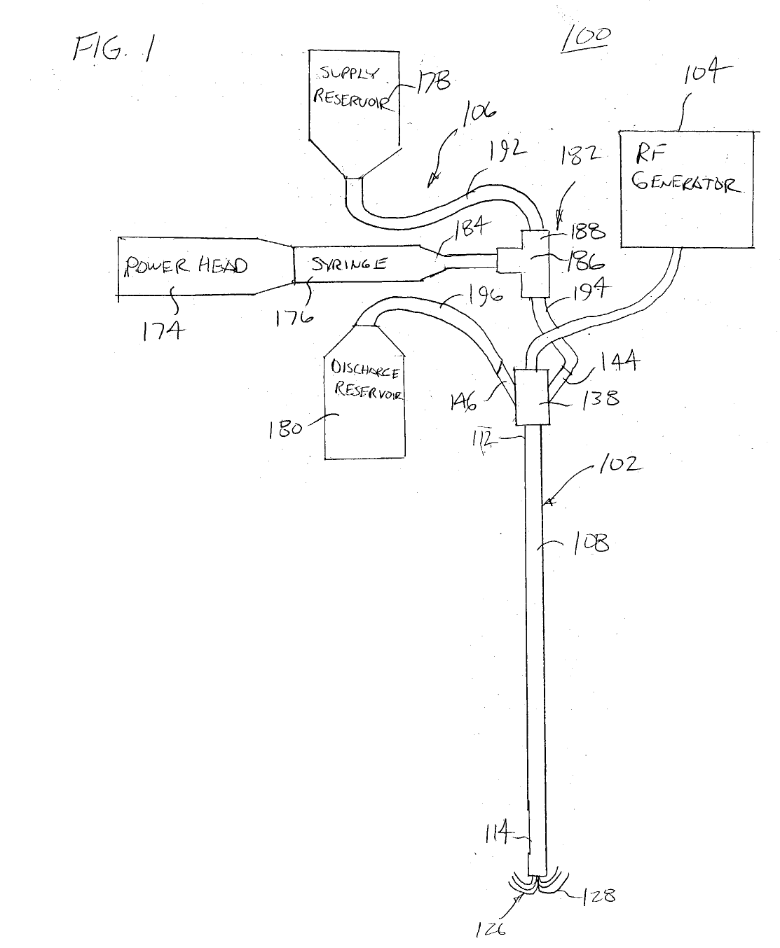

FIG. 1 illustrates a tissue ablation system 100 constructed in accordance with a preferred embodiment of the present inventions. The tissue ablation system 100 generally comprises a probe assembly 102 configured for introduction into the body of a patient for ablative treatment of target tissue, a radio frequency (RF) generator 104 configured for supplying RF energy to the probe assembly 102 in a controlled manner, and a pump assembly 106 configured for providing and circulating a coolant through the probe assembly 102, so that a more efficient and effective ablation treatment is effected.

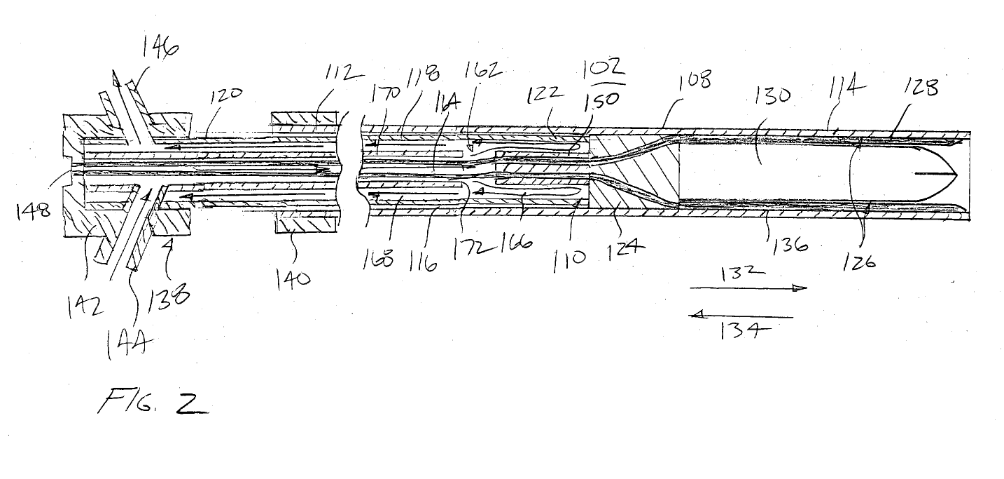

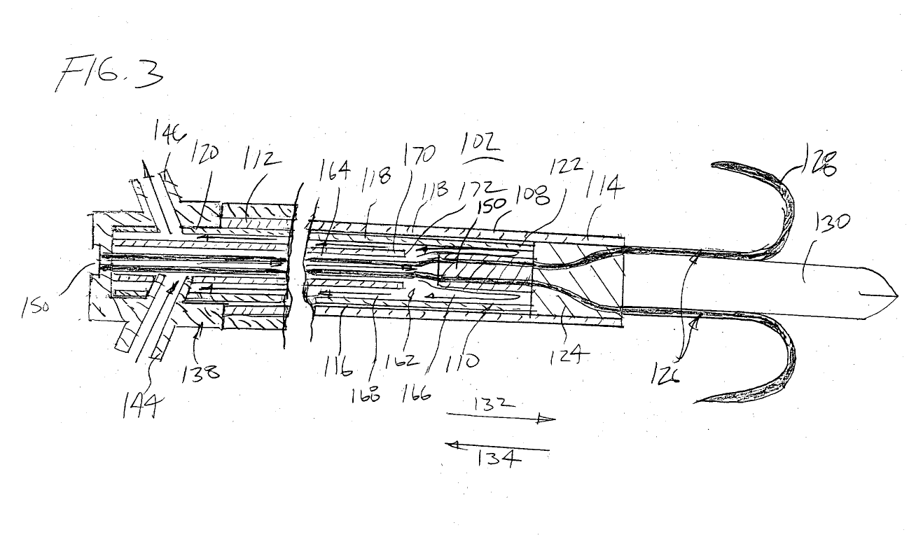

Referring specifically now to FIGS. 2 and 3, the probe assembly 102 generally comprises an elongated cannula 108 and an inner probe 110 slidably disposed within the cannula 108. As will be described in further detail below, the cannula 108 serves to deliver the active portion of the inner probe 110 to the target tissue. The cannula 108 has a proximal end 112, a distal end 114, and a central lumen 116 extending through the cannula 108 between the proximal end 112 and the distal end 114. As will be described in further detail below, the cannula 108 may be rigid, semi-rigid, or flexible depending upon the designed means for introducing the cannula 108 to the target tissue. The cannula 108 is composed of a suitable material, such as plastic, metal or the like, and has a suitable length, typically in the range from 5 cm to 30 cm, preferably from 10 cm to 20 cm. If composed of an electrically conductive material, the cannula 108 is preferably covered with an insulative material. The cannula 108 has an outside diameter consistent with its intended use, typically being from 1 mm to 5 mm, usually from 1.3 mm to 4 mm. The cannula 108 has an inner diameter in the range from 0.7 mm to 4 mm, preferably from 1 mm to 3.5 mm.

The inner probe 110 comprises a reciprocating shaft 118 having a proximal end 120 and a distal end 122, a cylindrical block 124 mounted to the distal end 114 of the shaft 118, a core member 130 mounted to the cylindrical block 124, and an array 126 of tissue penetrating needle electrodes 128 circumferentially disposed about the core member 130 and mounted within the cylindrical block 124. Like the cannula 108, the shaft 118, cylindrical block 124, and core member 130 are composed of a suitable material, such as plastic, metal or the like. It can be appreciated that longitudinal translation of the shaft 118 relative to the cannula 108 in a distal direction 132 deploys the core member 130 and electrode array 126 from the distal end 114 of the cannula 108 (FIG. 3), and longitudinal translation of the shaft 118 relative to the cannula 108 in a proximal direction 134 retracts the core member 130 and electrode array 126 into the distal end 114 of the cannula 108 (FIG. 2).

The core member 130 is disposed coaxially within the central lumen 116 of the cannula 108 to maintain substantially equal circumferential spacing between the needle electrodes 128 retracted in the central lumen 116. An annular envelope 136 is defined between the inner surface of the cannula 108 and the outer surface of the core member 130 when the core member 130 is retracted within the distal end 114 of the cannula 108. The width of the annular envelope 136 (defined by the distance between the outer surface of the core member 130 and the inner surface of the cannula 108) is typically in the range from 0.1 mm to 1 mm, preferably from 0.15 mm to 0.5 mm, and will usually be selected to be slightly larger than the thickness of the individual electrodes 128 in the radial direction. In this manner, when retracted within the cannula 108 (FIG. 2), the electrode array 126 is placed in a radially collapsed configuration, and the individual needle electrodes 128 are constrained and held in generally axially aligned positions within the cannula 108 over the outer cylindrical surface of the core member 130, to facilitate its introduction to the tissue target site.

Each of the individual needle electrodes 128 is in the form of a small diameter metal element, which can penetrate into tissue as it is advanced from a target site within the target region. When deployed from the cannula 108 (FIG. 3), the electrode array 126 is placed in a three-dimensional configuration that usually defines a generally ellipsoidal or spherical volume having a periphery with a maximum radius in the range from 0.5 to 3 cm. The needle electrodes 128 are resilient and pre-shaped to assume a desired configuration when advanced into tissue. In the illustrated embodiment, the needle electrodes 128 diverge radially outwardly from the cannula 108 in a uniform pattern, i.e., with the spacing between adjacent needle electrodes 128 diverging in a substantially uniform and/or symmetric pattern. In the illustrated embodiment, the needle electrodes 128 also evert proximally, so that they face partially or fully in the proximal direction 134 when fully deployed. In exemplary embodiments, pairs of adjacent needle electrodes 128 can be spaced from each other in similar or identical, repeated patterns and can be symmetrically positioned about an axis of the shaft 118. It will be appreciated that a wide variety of particular patterns can be provided to uniformly cover the region to be treated. It should be noted that a total of six needle electrodes 128 are illustrated in FIG. 1. Additional needle electrodes 128 can be added in the spaces between the illustrated electrodes 128, with the maximum number of needle electrodes 128 determined by the electrode width and total circumferential distance available (i.e., the needle electrodes 128 could be tightly packed).

Each individual needle electrode 128 is preferably composed of a single wire that is formed from resilient conductive metals having a suitable shape memory, such as stainless steel, nickel-titanium alloys, nickel-chromium alloys, spring steel alloys, and the like. The wires may have circular or non-circular cross-sections, but preferably have rectilinear cross-sections. In this manner, the needle electrodes 128 are generally stiffer in the transverse direction and more flexible in the radial direction. By increasing transverse stiffness, proper circumferential alignment of the needle electrodes 128 within the annular envelope 136 is enhanced. Exemplary needle electrodes will have a width (in the circumferential direction) in the range from 0.2 mm to 0.6 mm, preferably from 0.35 mm to 0.40 mm, and a thickness (in the radial direction) in the range from 0.05 mm to 0.3 mm, preferably from 0.1 mm to 0.2 mm.

The distal ends of the needle electrodes 128 may be honed or sharpened to facilitate their ability to penetrate tissue. The distal ends of these needle electrodes 128 may be hardened using conventional heat treatment or other metallurgical processes. They may be partially covered with insulation, although they will be at least partially free from insulation over their distal portions. It will be appreciated that as the core member 130 distally moves with the electrode array 126, it will enter the tissue at the same time as the electrode array 126. To enhance tissue penetration, the core member comprises a sharpened distal end. The proximal ends of the needle electrodes 128 may be directly coupled to the connector assembly (described below), or alternatively, may be indirectly coupled thereto via other intermediate electrical conductors, e.g., RF wires. Optionally, the shaft 118 and any component between the shaft 118 and the needle electrodes 128, are composed of an electrically conductive material, such as stainless steel, and may therefore conveniently serve as intermediate electrical conductors.

In the illustrated embodiment, the RF current is delivered to the electrode array 126 in a monopolar fashion, which means that current will pass from the electrode array 126, which is configured to concentrate the energy flux in order to have an injurious effect on the surrounding tissue, and a dispersive electrode (not shown), which is located remotely from the electrode array 126 and has a sufficiently large area (typically 130 cm² for an adult), so that the current density is low and non-injurious to surrounding tissue. In the illustrated embodiment, the dispersive electrode may be attached externally to the patient, e.g., using a contact pad placed on the patient's flank. In a monopolar arrangement, the needle electrodes 128 are bundled together with their proximal portions having only a single layer of insulation over the cannula 108.

Alternatively, the RF current is delivered to the electrode array 126 in a bipolar fashion, which means that current will pass between “positive” and “negative” electrodes 128 within the array 126. In a bipolar arrangement, the positive and negative needle electrodes 128 will be insulated from each other in any regions where they would or could be in contact with each other during the power delivery phase.

Optionally, the core member 130 may be electrically coupled to the electrode array 126, in which case it acts as an additional needle electrode 128 of the same polarity as the electrodes 128, or may be electrically isolated from the electrodes 128. When the core member 130 is electrically isolated, it can remain neutral during a treatment protocol, or alternatively it may be energized in the opposite polarity, and thus acts as a return electrode in a bipolar arrangement.

Further details regarding needle electrode array-type probe arrangements are disclosed in U.S. Pat. No. 6,379,353, entitled “Apparatus and Method for Treating Tissue with Multiple Electrodes,” which is hereby expressly incorporated herein by reference.

The probe assembly 102 further comprises a connector assembly 138, which includes a connector sleeve 140 mounted to the proximal end 112 of the cannula 108 and a connector member 142 slidably engaged with the sleeve 140 and mounted to the proximal end 120 of the shaft 118. The connector member 142 of the connector assembly 138 comprises an inlet fluid port 144 and an outlet fluid port 146. The connector member 142 also comprises an electrical connector 148 in which the proximal ends of the needle electrodes 128 (or alternatively, intermediate conductors) extending through the shaft 118 of the inner probe 110 are coupled. The connector assembly 138 can be composed of any suitable rigid material, such as, e.g., metal, plastic, or the like.

The probe assembly 102 further comprises a heat sink 150 mounted within the distal end 114 of the shaft 118. The heat sink 150 is thermally coupled to the electrode array 126 and serves to thermally draw heat away from the electrode array 126 during RF ablation.

In the illustrated embodiment, the heat sink 150 is composed of a solid piece of thermally conductive material, such as stainless steel, nickel titanium, aluminum or copper. In this manner, the local temperature of the tissue adjacent the electrode array 126 is reduced, thereby minimizing tissue charring and vaporization.

In the illustrated embodiment, needle electrodes 128 extend through the heat sink 150, and back through the lumen of an inner tube (described below) to the electrical connector 148 of the connector assembly 138. Alternatively, the proximal ends of the needle electrodes 128 are embedded into the distal end of the heat sink 150, in which case, intermediate electrical conductors (such as RF wires) will be connected between the needle electrodes 128 and the electrical connector 148 of the connector assembly 138. If the shaft 118 and cylindrical block 124 serve as intermediate conductors, the proximal ends of the needle electrodes 128 may be welded to the distal end of the heat sink 150.

Referring to FIG. 4, an alternative embodiment of a heat sink 151 can be used in place of the solid heat sink 150. The heat sink 151 comprises a cylindrical member 152 having a sealed cavity 154 formed therein. A medium 156 that is normally in a liquid state in the absence of ablative thermal energy is disposed within the sealed cavity 154. The liquid medium 156 preferably has a relatively low boiling point, e.g., less than the boiling point of distilled water. For example, alcohol can be used as the liquid medium. The air pressure within the sealed cavity 154 is less than atmospheric pressure (i.e., the air pressure outside of the sealed cavity 154), and preferably, is under a vacuum. Thus, because the liquid medium 156 is subjected to the vacuum, its boiling point is much lower than if it were subjected to atmospheric pressure.

It will thus be appreciated that as thermal energy is conducted from the electrode array 126 to the heat sink 150, the sealed cavity 154 heats up, causing the liquid medium 156 to boil and transition to a gaseous state. As result, thermal energy is quickly absorbed by the medium 156 when it transitions from a liquid state to a gaseous state, which is then released when the medium 156 cools and transitions back from the gaseous state to the liquid state. So that the heated gaseous medium 156 flows away from the electrode array 126 (i.e., from the distal end to the proximal end of the heat sink 151), and the cooled liquid medium 156 flows towards the electrode array 126 (i.e., from the proximal end to the distal end of the heat sink 151) in a stable and controlled manner (as shown by arrows 160), the heat sink 151 contains a wicking material 158, such as, e.g., woven stainless steel.

Referring back to FIGS. 2 and 3, the probe assembly 102 further comprises a coolant flow conduit 162 that is in fluid communication with the heat sink 150 and serves to thermally draw heat away from the heat sink 150, thereby maximizing the cooling effect that the heat sink 150 has on the electrode array 126. The coolant flow conduit 162 comprises a cooling lumen 164, a thermal exchange cavity 166, and a return lumen 168. In the illustrated embodiment, the cooling and return lumens 164 and 168 are coaxial and are formed by disposing an inner tube 170 within the shaft 118. Specifically, the inner tube 170 comprises an open distal end 172 that resides proximal to the heat sink 150. The inner tube 170 comprises a central lumen, which serves as cooling lumen 164, and is in fluid communication with the inlet fluid port 144. An annular lumen, which is formed between the outer surface of the inner tube 170 and the inner surface of the shaft 118, serves as the return lumen 168 and is in fluid communication with the outlet fluid port 146 on the connector assembly 138.

Alternatively, the central lumen of the inner tube 170 can serve as the return lumen 168, and the annular lumen between the inner tube 170 and the shaft 118 can serve as the cooling lumen 164. More alternatively, the cooling and return lumens 164 and 168 are not coaxial, but rather are disposed within the shaft 118 in a side-by-side relationship.

In any event, the thermal exchange cavity 166 is disposed within the distal end of the shaft 118 and surrounds the heat sink 150. The thermal exchange cavity 166 is in fluid communication with the distal ends of the cooling and return lumens 164 and 168. Thus, it will be appreciated that the cooling lumen 164 is configured to convey a cooled medium, such as, e.g., saline, into the thermal exchange cavity 166, thereby cooling the heat sink 150, and the return lumen 168 is configured to convey the resultant heated medium from the thermal exchange cavity 166 (path of medium shown by arrows). It should be noted that for the purposes of this specification, a cooled medium is any medium that has a temperature suitable for drawing heat away from the heat sink in which the coolant flow conduit 162 is in communication with. For example, a cooled medium at room temperature or lower is well suited for cooling the heat sink.

Referring back to FIG. 1, the RF generator 104 is electrically connected to the electrical connector 148 of the connector assembly 138, which as previously described, is directly or indirectly electrically coupled to the electrode array 126. The RF generator 104 is a conventional RF power supply that operates at a frequency in the range from 200 KHz to 1.25 MHz, with a conventional sinusoidal or non-sinusoidal wave form. Such power supplies are available from many commercial suppliers, such as Valleylab, Aspen, and Bovie. Most general purpose electrosurgical power supplies, however, operate at higher voltages and powers than would normally be necessary or suitable for vessel occlusion. Thus, such power supplies would usually be operated at the lower ends of their voltage and power capabilities. More suitable power supplies will be capable of supplying an ablation current at a relatively low voltage, typically below 150V (peak-to-peak), usually being from 50V to 100V. The power will usually be from 20 W to 200 W, usually having a sine wave form, although other wave forms would also be acceptable. Power supplies capable of operating within these ranges are available from commercial vendors, such as Radio Therapeutics of San Jose, Calif., who markets these power supplies under the trademarks RF2000™ (100 W) and RF3000™ (200W).

The pump assembly 106 comprises a power head 174 and a syringe 176 that is front-loaded on the power head 174 and is of a suitable size, e.g., 200 ml. The power head 174 and the syringe 176 are conventional and can be of the type described in U.S. Pat. No. 5,279,569 and supplied by Liebel-Flarsheim Company of Cincinnati, Ohio. The pump assembly 106 further comprises a source reservoir 178 for supplying the cooling medium to the syringe 176, and a collection reservoir 180 for collecting the heated medium from the probe assembly 102. The pump assembly 106 further comprises a tube set 182 removably secured to an outlet 184 of the syringe 176. Specifically, a dual check valve 186 is provided with first and second legs 188 and 190, of which the first leg 188 serves as a liquid inlet connected by tubing 192 to the source reservoir 178. The second leg 190 is an outlet leg and is connected by tubing 194 to the inlet fluid port 144 on the connector assembly 138. The collection reservoir 180 is connected to the outlet fluid port 146 on the connector assembly 138 via tubing 196.

Thus, it can be appreciated that the pump assembly 106 can be operated to periodically fill the syringe 176 with the cooling medium from the source reservoir 178, and convey the cooling medium from the syringe 176, through the tubing 194, and into the inlet fluid port 144 on the connector assembly 138. Heat medium is conveyed from the outlet fluid port 146 on the connector assembly 138, through the tubing 196, and into the collection reservoir 180. The pump assembly 106, along with the RF generator 104, can include control circuitry to automate or semi-automate the cooled ablation process. Further details on the structure and operation of a controlled RF generator/pump assembly suitable for use with the tissue ablation system 100 are disclosed in U.S. Pat. No. 6,235,022, entitled “RF generator and pump apparatus and system and method for cooled ablation,” which is hereby fully and expressly incorporated herein by reference. A commercial embodiment of such an assembly is marketed as the Model 8004 RF generator and Pump System by Cardiac Pathways, Inc., located in San Jose, Calif.

Having described the structure of the tissue ablation system 100, its operation in treating targeted tissue will now be described. The treatment region may be located anywhere in the body where hyperthermic exposure may be beneficial. Most commonly, the treatment region will comprise a solid tumor within an organ of the body, such as the liver, kidney, pancreas, breast, prostrate (not accessed via the urethra), and the like. The volume to be treated will depend on the size of the tumor or other lesion, typically having a total volume from 1 cm³ to 150 cm³, and often from 2 cm³ to 35 cm³. The peripheral dimensions of the treatment region may be regular, e.g., spherical or ellipsoidal, but will more usually be irregular. The treatment region may be identified using conventional imaging techniques capable of elucidating a target tissue, e.g., tumor tissue, such as ultrasonic scanning, magnetic resonance imaging (MRI), computer-assisted tomography (CAT), fluoroscopy, nuclear scanning (using radiolabeled tumor-specific probes), and the like. Preferred is the use of high resolution ultrasound of the tumor or other lesion being treated, either intraoperatively or externally.

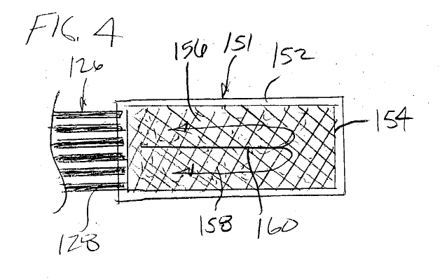

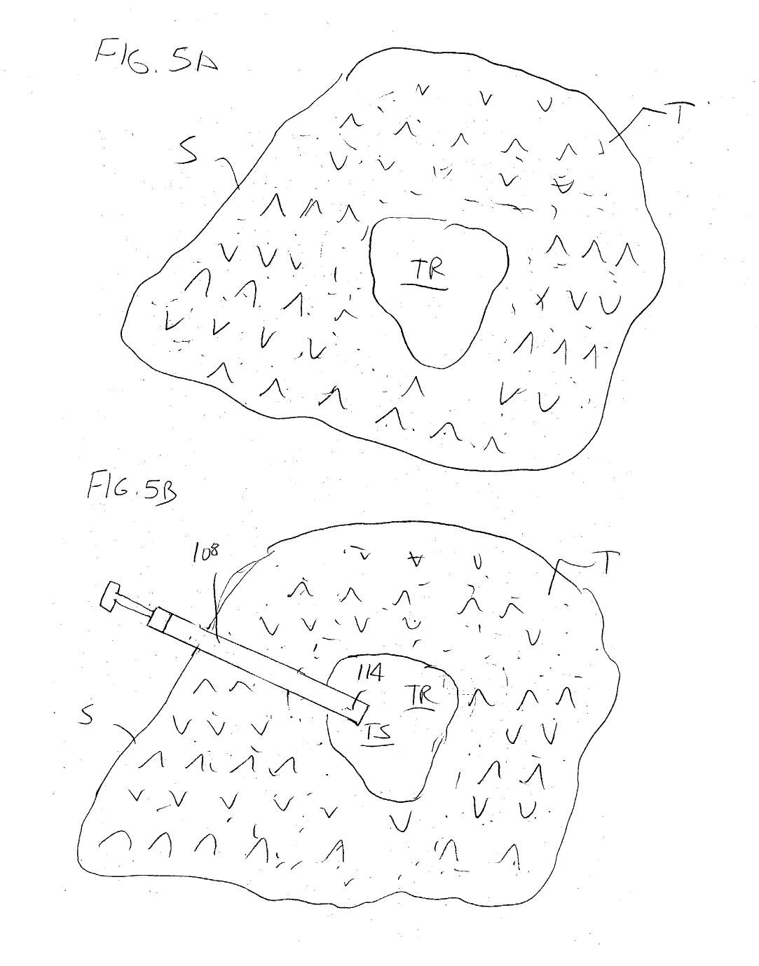

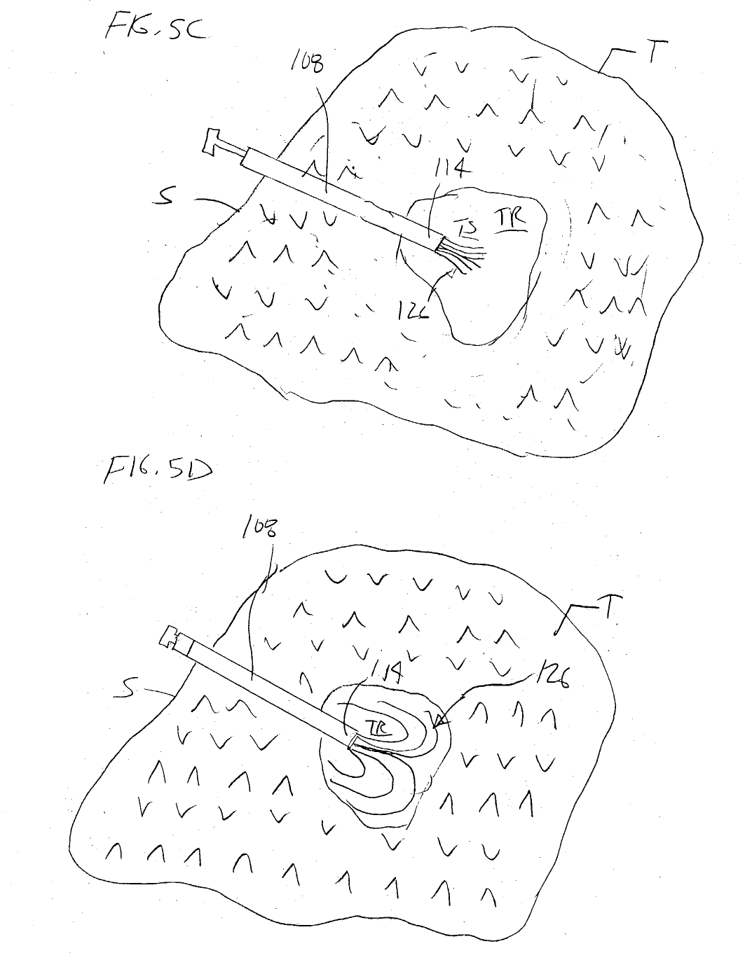

Referring now to FIGS. 5A-5D, the operation of the tissue ablation system 100 is described in treating a treatment region TR within tissue T located beneath the skin or an organ surface S of a patient. The tissue T prior to treatment is shown in FIG. 5A. The cannula 108 is first introduced within the treatment region TR, so that the distal end 114 of the cannula 108 is located at the target site TS, as shown in FIG. 5B. This can be accomplished using any one of a variety of techniques. In some cases, the cannula 108 and inner probe 110 may be introduced to the target site TS percutaneously directly through the patient's skin or through an open surgical incision. In this case, the cannula 108 may have a sharpened tip, e.g., in the form of a needle, to facilitate introduction to the treatment region TR. In such cases, it is desirable that the cannula 108 or needle be sufficiently rigid, i.e., have a sufficient column strength, so that it can be accurately advanced through tissue T. In other cases, the cannula 108 may be introduced using an internal stylet that is subsequently exchanged for the shaft 118 and electrode array 126. In this latter case, the cannula 108 can be relatively flexible, since the initial column strength will be provided by the stylet. More alternatively, a component or element may be provided for introducing the cannula 108 to the target site TS. For example, a conventional sheath and sharpened obturator (stylet) assembly can be used to initially access the tissue T. The assembly can be positioned under ultrasonic or other conventional imaging, with the obturator/stylet then removed to leave an access lumen through the sheath. The cannula 108 and inner probe 110 can then be introduced through the sheath lumen, so that the distal end 114 of the cannula 108 advances from the sheath into the target site TS.

After the cannula 108 is properly placed, the shaft 118 is distally advanced to deploy the electrode array 126 radially outward from the distal end 114 of the cannula 108, as shown in FIG. 5C. The shaft 118 will be advanced sufficiently, so that the electrode array 126 fully everts in order to circumscribe substantially the entire treatment region TR, as shown in FIG. 5D. The sharpened end of the core member 130 facilitates introduction of the electrode array 126 within the treatment region TR.

The RF generator 104 is then connected to the connector assembly 138 via the electrical connector 148 and the pump assembly 106 is connected to the connector assembly 138 via the fluid ports 144 and 146, and then operated to ablate the treatment region TR.

During the RF ablation process, the pump assembly 106 is operated to cool the electrode array 126. Specifically, the power head 174 conveys the cooled medium from the syringe 176 under positive pressure, through the tubing 194, and into the inlet fluid port 144 on the connector assembly 138. The cooled medium then travels through the cooling lumen 164 and into the thermal exchange cavity 166 adjacent the heat sink 150. Thermal energy is transferred from the heat sink 150 to the cooled medium, thereby cooling the heat sink (and thus the electrode array 126) and heating the medium. The heated medium is then conveyed from the thermal exchange cavity 166 back through the return lumen 168. From the return lumen 168, the heated medium travels through the outlet fluid port 146 on the connector assembly 138, through the tubing 196, and into the collection reservoir 180. This process is continued during the ablation process.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A medical probe assembly for ablating tissue, comprising:
an elongated shaft having a proximal end and a distal end;
one or more needle electrodes extending from the distal end of the shaft;
a heat sink disposed within the distal end of the shaft in thermal communication with the one or more needle electrodes; and
a coolant flow conduit disposed within the shaft in fluid communication with the heat sink.

2. The medical probe assembly of claim 1, wherein the elongated shaft is a surgical probe shaft.

3. The medical probe assembly of claim 1, wherein one or more needle electrodes comprises an array of needle electrodes.

4. The medical probe assembly of claim 3, further comprising a core member extending from the distal end of the shaft, wherein the needle electrode array is circumferentially disposed about the core member.

5. The medical probe assembly of claim 3, wherein the needle electrode array everts proximally.

6. The medical probe assembly of claim 1, further comprising one or more radio frequency (RF) wires coupled to the one or more needle electrodes.

7. The medical probe assembly of claim 1, wherein the heat sink is completely solid.

8. The medical probe assembly of claim 1, wherein the heat sink comprises:
a sealed cavity having an internal air pressure that is lower than an external air pressure; and
a medium disposed within the sealed cavity, wherein the medium transitions from a liquid state to a gaseous state when heated, and transitions from the gaseous state back to the liquid state when cooled.

9. The medical probe assembly of claim 8, wherein the heat sink further comprises a wicking material disposed within the sealed cavity.

10. The medical probe assembly of claim 8, wherein the liquid medium has a boiling point that is less than the boiling point of water.

11. The medical probe assembly of claim 1, wherein the coolant flow conduit comprises a cooling lumen for conveying a cooled medium from the proximal end of the shaft to the heat sink, and a return lumen for conveying a heated medium from the heat sink to the proximal end of the shaft.

12. The medical probe assembly of claim 11, wherein the coolant flow conduit further comprises a thermal exchange cavity in fluid communication between the cooling and return lumens and the heat sink.

13. The medical probe assembly of claim 11, further comprising an inner tube disposed within the shaft, wherein one of the cooling lumen and return lumen is formed within the inner tube, and the other of the cooling lumen and return lumen is an annular lumen formed between an inner surface of the shaft and an outer surface of the inner tube.

14. The medical probe assembly of claim 13, wherein the cooling lumen is formed within the inner tube, and the return lumen is formed the annular lumen formed between the inner surface of the shaft and the outer surface of the inner tube.

15. The medical probe assembly of claim 1, further comprising a cannula having a central lumen, wherein the shaft is reciprocally disposed within the central lumen of the cannula.

16. A medical probe assembly for ablating tissue, comprising:
an elongated shaft having a proximal end and a distal end;
an array of needle electrodes extending from the distal end of the shaft;
a heat sink disposed within the distal end of the shaft in thermal communication with the needle electrode array;
a thermal exchange cavity in fluid communication with the heat sink;
a cooling lumen for conveying a cooled medium from the proximal end of the shaft to the thermal exchange cavity; and
a return lumen for conveying a heated medium from the thermal exchange cavity to the proximal end of the shaft.

17. The medical probe assembly of claim 16, wherein the elongated shaft is a surgical probe shaft.

18. The medical probe assembly of claim 16, further comprising a core member extending from the distal end of the shaft, wherein the needle electrode array is circumferentially disposed about the core member.

19. The medical probe assembly of claim 16, wherein the needle electrode array everts outward.

20. The medical probe assembly of claim 16, further comprising one or more radio frequency (RF) wires coupled to the needle electrode array.

21. The medical probe assembly of claim 16, wherein the heat sink is completely solid.

22. The medical probe assembly of claim 16, wherein the heat sink comprises:
a sealed cavity having an internal air pressure that is lower than an external air pressure; and
a medium disposed within the sealed cavity, wherein the medium transitions from a liquid state to a gaseous state when heated, and transitions from the gaseous state back to the liquid state when cooled.

23. The medical probe assembly of claim 22, wherein the heat sink further comprises a wicking material disposed within the sealed cavity.

24. The medical probe assembly of claim 22, wherein the liquid medium has a boiling point that is less than the boiling point of water.

25. The medical probe assembly of claim 16, further comprising an inner tube disposed within the shaft, wherein one of the cooling lumen and return lumen is formed within the inner tube, and the other of the cooling lumen and return lumen is an annular lumen formed between an inner surface of the shaft and an outer surface of the inner tube.

26. The medical probe assembly of claim 25, wherein the cooling lumen is formed within the inner tube, and the return lumen is the annular lumen.

27. The medical probe assembly of claim 16, further comprising a cannula having a central lumen, wherein the shaft is reciprocally disposed within the central lumen of the cannula.

28. A tissue ablation system, comprising:
an elongated shaft having a proximal end and a distal end;
one or more needle electrodes extending from the distal end of the shaft;
a heat sink disposed within the distal end of the shaft in thermal communication with the one or more needle electrodes;
a coolant flow conduit in fluid communication with the heat sink;
an ablation source operably coupled to the one or more needle electrodes; and
a pump assembly operably coupled to the coolant flow conduit.

29. The tissue ablation system of claim 28, wherein the elongated shaft is a surgical probe shaft.

30. The tissue ablation system of claim 28, wherein one or more needle electrodes comprises an array of needle electrodes.

31. The tissue ablation system of claim 30, further comprising a core member extending from the distal end of the shaft, wherein the needle electrode array is circumferentially disposed about the core member.

32. The tissue ablation system of claim 30, wherein the needle electrode array everts proximally.

33. The tissue ablation system of claim 28, wherein the ablation source is an radio frequency (RF) ablation source, and further comprising one or more RF wires coupled between the one or more needle electrodes and the RF ablation source.

34. The tissue ablation system of claim 28, wherein the heat sink is completely solid.

35. The tissue ablation system of claim 28, wherein the heat sink comprises:
a sealed cavity having an internal air pressure that is lower than an external air pressure; and
a medium disposed within the sealed cavity, wherein the medium transitions from a liquid state to a gaseous state when heated, and transitions from the gaseous state back to the liquid state when cooled.

36. The tissue ablation system of claim 35, wherein the heat sink further comprises a wicking material disposed within the sealed cavity.

37. The tissue ablation system of claim 35, wherein the liquid medium has a boiling point that is less than the boiling point of water.

38. The tissue ablation system of claim 28, wherein the coolant flow conduit comprises a cooling lumen for conveying a cooled medium from the proximal end of the shaft to the heat sink, and a return lumen for conveying a heated medium from the heat sink to the proximal end of the shaft.

39. The tissue ablation system of claim 38, wherein the coolant flow conduit further comprises a thermal exchange cavity in fluid communication between the cooling and return lumens and the heat sink.

40. The tissue ablation system of claim 38, further comprising an inner tube disposed within the shaft, wherein one of the cooling lumen and return lumen is formed within the inner tube, and the other of the cooling lumen and return lumen is an annular lumen formed between an inner surface of the shaft and an outer surface of the inner tube.

41. The tissue ablation system of claim 40, wherein the cooling lumen is formed within the inner tube, and the return lumen is formed the annular lumen formed between the inner surface of the shaft and the outer surface of the inner tube.

42. The tissue ablation system of claim 28, further comprising a cannula having a central lumen, wherein the shaft is reciprocally disposed within the central lumen of the cannula.
